(19) United States
(12) Patent Application Publication
(10) Pub. No.: US 2011/0237942 A1
(43) Pub. Date: Sep. 29, 2011

(54) BIOIMAGING METHOD USING NEAR-INFRARED (NIR) FLUORESCENT MATERIAL

(76) Inventors: Tamotsu Zako, Saitama (JP); Mizuo Maeda, Saitama (JP); Kohei Soga, Chiba (JP); Hidehiro Kishimoto, Chiba (JP); Hiroshi Hyodo, Chiba (JP); Masaaki Ito, Chiba (JP); Kazuhiro Kaneko, Chiba (JP)

(21) Appl. No.: 13/072,275

(22) Filed: Mar. 25, 2011

Related U.S. Application Data
(60) Provisional application No. 61/317,442, filed on Mar. 25, 2010.

(51) Int. Cl. A61B 6/00; G01J 1/58

(52) U.S. Cl. 600/431; 250/458.1

(57) ABSTRACT

This invention provides a novel bioimaging technique that can achieve a deep observation depth and a novel method for marking a lesion that allows clear recognition of the lesion from outside a living body. This invention also provides a bioimaging marker comprising a fluorescent material obtained by doping a ceramic with rare earths and the like and a bioimaging technique comprising detecting near-infrared fluorescence that can sufficiently penetrate a living body generated upon excitation of the marker with near-infrared excitation light.

### CROSS REFERENCE TO RELATED APPLICATIONS

This non-provisional application claims the benefit under 35 USC §119(e) of U.S. Provisional Application No. 61/317,442 filed on Mar. 25, 2010. The entire contents of the above application is hereby incorporated by reference into the present application.

### BACKGROUND OF THE INVENTION

1. Field of the Invention

The present invention relates to a bioimaging marker comprising a fluorescent material obtained by doping a ceramic with rare earths and the like. The present invention also relates to a bioimaging system and a bioimaging method using the bioimaging marker.

2. Background Art

Bioimaging techniques have been gaining attention recently as tools for observation of biological phenomena both in vivo and in vitro in the field of biomedical research. In particular, there have been many attempts to apply near-infrared (hereafter referred to as “NIR”) light to biomedical photonics within the wavelength range from 800 to 2000 nm in which NIR light can sufficiently penetrate a living body.

In recent years, bioimaging techniques using upconverting phosphors (hereafter referred to as “UCPs”) have been developed (Kamimura, M. et. al., Langmuir 24, 8864-70, (2008); Lim, S. F. et. al., Nano Lett 6, 169-74, (2006); Prasad, P. N., Crystals and Liquid Crystals 415, 1-7, (2004); Sivakumar, S. et. al., Chemistry-a European J. 12, 5878-5884, (2006); Zako, T. et. al., Journal of Materials Science 43, 5325-5330, (2008); Zako, T. et. al., Biochem Biophys Res Commun 381, 54-8, (2009); and Zijlmans, H. J. et. al., Anal Biochem 267, 30-6 (1999)). UCPs are ceramics doped with rare earth ions. They emit visible light as a result of upconversion luminescence upon excitation with NIR light (so-called “NIR-VIS imaging”) (Auzel, F., Chem Rev 104, 139-73, (2004)).

In the case of NIR-VIS imaging, NIR light used as excitation light can deeply penetrate a living body because of its low degree of scattering. However, it has been difficult to detect visible light generated as a result of upconversion luminescence from a site deep within a living body because of the influence of light scattering. Therefore, the observation depth is shallow in cases of NIR-VIS imaging techniques, which has been significantly problematic. Accordingly, a novel bioimaging technique that can achieve a deep observation depth has been awaited in the art.

In addition, at present, a lesion to be resected is detected by an endoscopic operation, and marking of such a lesion is carried out by tattoo injection for surgery for treatment of cancer such as colon/rectal cancer. However, in this case, tattoo injection is performed inside the colon or rectal wall (i.e., on the mucosal layer). Thus, it may be difficult to identify the site marked by tattoo injection from outside the colon/rectum (i.e., from the serosal side) during surgery due to dispersion of ink or fake tattoo. Therefore, it is impossible to clearly determine the lesion area during surgery, requiring resection of an organ/tissue area greater than the actual lesion area. This imposes significant burdens on patients.

Therefore, a novel marking method that allows clear determination of a lesion even from the serosal side has been awaited in the art.

### SUMMARY OF THE INVENTION

The present invention provides a novel bioimaging technique using NIR light that can achieve a deep observation depth and a novel method for marking a lesion that allows clear recognition of the lesion from outside a living body.

As a result of intensive studies in order to solve the above problems, the present inventors found that a fluorescent material obtained by doping a ceramic with rare earths and the like emits NIR fluorescence that can sufficiently penetrate a living body upon excitation with NIR excitation light that can sufficiently penetrate a living body. This has led to the completion of the present invention.

Specifically, the present invention encompasses the following inventions.

[1] A bioimaging marker comprising a fluorescent material obtained by doping a ceramic with one or more rare earth ions and/or one or more elemental ions selected from the group consisting of uranium (U), titanium (Ti), chromium (Cr), nickel (Ni), manganese (Mn), molybdenum (Mo), rhenium (Re), and osmium (Os) ions, wherein the marker is in the form of any one of the following (a) to (c):

(a) a clip comprising a fluorescent material;

(b) an ink solution containing a fluorescent material; or

(c) a probe capable of recognizing a particular biomolecule to which a fluorescent material is bound, and wherein

the marker emits near-infrared fluorescence at 1000 to 2000 nm when irradiated with near-infrared excitation light at 780 to 1700 nm.

[2] The marker according to [1], wherein the clip comprise the fluorescent material in the arm.
[3] The marker according to [1] or [2], wherein the fluorescent material is in the form of a nanoparticle of yttrium oxide obtained by codoping of Y₂O₃ with ytterbium (Yb) ion and erbium (Er) ion.
[4] The marker according to [3], which emits near-infrared fluorescence at 1430 to 1670 nm when irradiated with near-infrared excitation light at 900 to 1000 nm.
[5] A bioimaging system for visualizing a marker introduced into a living body with the use of near-infrared light, which comprises at least the following (i) to (iv):

(i) the marker according to any one of [1] to [4], which is introduced into a living body;

(ii) a light source for irradiating the marker with near-infrared excitation light at 780 to 1700 nm from outside a living body;

(iii) a photographing means for detecting near-infrared fluorescence at 1000 to 2000 nm emitted from the marker excited by the light source, thereby obtaining image data; and

(iv) an image displaying means for displaying an observation image of image data obtained by the photographing means.

[6] The system according to [5], wherein the marker is irradiated with near-infrared excitation light at 900 to 1000 nm.
[7] The system according to [5] or [6], wherein the photographing means detects near-infrared fluorescence emitted from the marker at 1430 to 1670 nm.
[8] A bioimaging method using a marker introduced into a living body of an animal wherein the bioimaging system according to any one of [5] to [7] is used, which comprises the following steps of:

(a) introducing a marker into a living body of an animal;

(b) irradiating the marker from outside the living body with near-infrared excitation light from a light source; and

(c) detecting near-infrared fluorescence emitted from the excited fluorescent material by a photographing means.

[9] A bioimaging method using a marker introduced into a human organ or tissue wherein the bioimaging system according to any one of [5] to [7] is used, which comprises the following steps of:

(a) irradiating a marker introduced into a human organ or tissue from outside the human organ or tissue with near-infrared excitation light from a light source; and

(b) detecting near-infrared fluorescence emitted by the excited fluorescent material by a photographing means.

### EFFECTS OF THE INVENTION

According to the present invention, a novel bioimaging technique that can achieve a deep observation depth and a novel method for marking a given site in a living body or a lesion that allows clear recognition of a marker from outside a living body can be provided.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1 (A) shows an FE-SEM image of Y₂O₃:YbEr-NP and FIG. 1 (B) shows XRD patterns.

FIG. 2 (A) shows an absorption spectrum of Y₂O₃:YbEr-NP, FIG. 2 (B) shows an energy level diagram of Y₂O₃:YbEr-NP, and FIG. 2 (C) shows fluorescence spectra of Y₂O₃:YbEr-NP (solid line) and Y₂O₃:Er-NP (dashed line).

FIG. 3 shows an optical absorption loss spectrum for a swine intestine. The solid line represents an optical absorption loss spectrum for the swine intestine, the dashed line represents an absorption spectrum for a water, and the single-dot chain line represents a fluorescence spectrum of Y₂O₃:YbEr-NP.

FIGS. 4 (A) to (C) show NIR images of markers comprising Y₂O₃:YbEr-NP in different forms, each of which was introduced into a swine intestine: (A) a Y₂O₃:YbEr-NP tablet; (B) an NIR clip; and (C) an NIR ink solution.

FIGS. 5 (A) and (B) each show U87MG cell detection results obtained using a Y₂O₃:YbEr-NP-bound probe: (A): a visible light image; and (B): an NIR image.

FIG. 6 schematically shows a near-infrared camera to which a surgical laparoscope is connected.

FIG. 7 (A) shows a visible light image and an NIR image of a Y₂O₃:YbEr-NP tablet positioned outside a swine colon sample and FIG. 7 (B) shows a visible light image and an NIR image of a Y₂O₃:YbEr-NP tablet positioned inside a swine colon sample.

FIG. 8 (A) shows photographs of an NIR clip (1) and an NIR clip (2). FIG. 8 (B) schematically shows an NIR clip fixed to a tissue.

FIG. 9 shows a visible light image and an NIR image of an NIR clip (1) (b) and those of an NIR clip (2) (a) positioned inside a swine colon sample.

FIG. 10 (A) shows the outline of surgical simulation for fixing an NIR clip (2) inside the large intestine of a pig via the transanal route using an endoscope. FIG. 10 (B) shows a visible light image of the NIR clip (2) fixed inside the colon using an endoscope and an NIR image from outside the colon using NIR camera attached to laparoscopy.

### DESCRIPTION OF THE PREFERRED EMBODIMENTS

The “fluorescent material” of the present invention can be obtained by doping a ceramic with one or more type(s) of rare earth ions. The term “ceramic” refers to a calcined product of oxysulfide, oxyhalide, fluoride, gallate, silicate, germanate, phosphate, or borate (but it is not limited thereto). Examples thereof include calcined products of yttrium oxide (Y₂O₃), lanthanum chloride (LaCl₃), lanthanum fluoride (LaF₃), strontium fluoride (SrF₂), yttrium alminate (YAlO₃), and yttrium aluminum garnet (Y₃Al₅O₁₂). Examples of “rare earths” include praseodymium (Pr), neodymium (Nd), gadolinium (Gd), dysprosium (Dy), holmium (Ho), erbium (Er), thulium (Tm), and ytterbium (Yb). The term “more types” used herein refers to two or more types. Instead of or in addition to the rare earth ions, ions of uranium (U), titanium (Ti), chromium (Cr), nickel (Ni), manganese (Mn), molybdenum (Mo), rhenium (Re), and osmium (Os) can be used as dopants. A fluorescent material can be obtained by doping a ceramic with one or more rare earth ions by a known method (Zako, T. et al., Biochem Biophys Res Commun 381, 54-8 (2009)).

The fluorescent material has a particle size of approximately 100 to 200 nm and preferably 130±25 nm. Fluorescent material particles used in the present invention may not have uniform particle sizes.

It is preferable to use a fluorescent material that emits NIR fluorescence at 1000 to 2000 nm upon excitation with NIR excitation light at 780 to 1700 nm, preferably 900 to 1000 nm, and particularly preferably 980 nm. This wavelength range is called the “biological window.” In this range, since water and biological tissue have minimal light absorbance and exhibit minimal autofluorescence, NIR fluorescence emitted by a fluorescent material can be easily detected even from outside a living body. Further, as explained in detail in the Examples described below, water molecules absorb light at 1420 nm. Therefore, it is better not to use a fluorescent material that emits NIR fluorescence at such wavelength. The wavelength range of NIR fluorescence emitted by a fluorescent material can vary depending on the dopant type and the ceramic type. Therefore, persons skilled in the art can adequately prepare or select a fluorescent material that emits NIR fluorescence within a desired wavelength range upon excitation with NIR excitation light at 780 to 1700 nm, preferably 900 to 1000 nm, and particularly preferably 980 nm with the use of a combination of an adequate ceramic and an adequate dopant.

According to the present invention, a fluorescent material is preferably a nanoparticle of Y₂O₃ codoped with Yb and Er ions (hereafter referred to as “Y₂O₃:YbEr-NP”). Such fluorescent material emits NIR fluorescence at 1430 to 1670 nm and preferably 1550 nm upon excitation with NIR excitation light at 780 to 1700 nm, preferably 900 to 1000 nm, and particularly preferably 980 nm.

In the present invention, a fluorescent material is contained in a marker in any form selected from among (a) to (c) described below.

### (a) A Clip Containing a Fluorescent Material

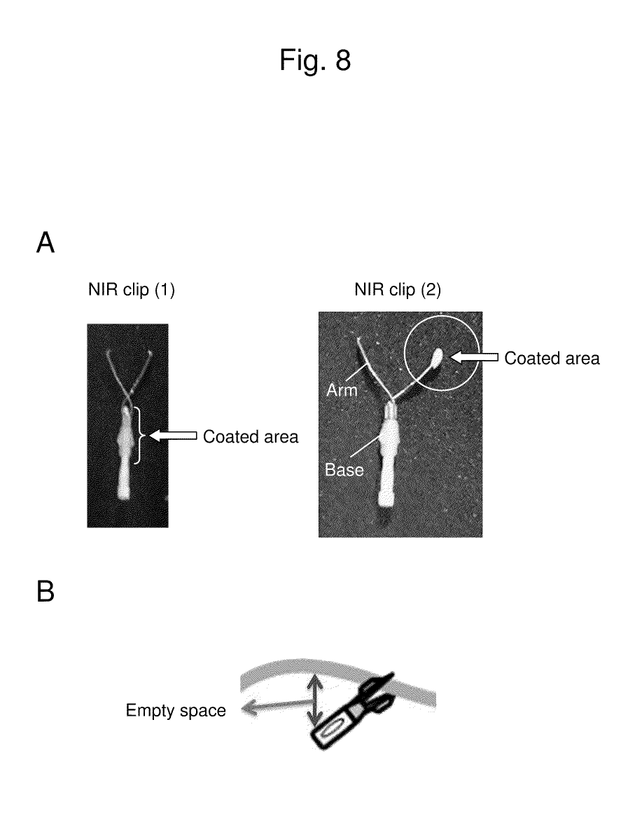

The clip of the present invention is in the shape of a clip generally used in the medical field and handled under endoscopy. The clip surface is partially or entirely coated with the above fluorescent material, or the clip partially or entirely contains the fluorescent material. Coating of a clip with a fluorescent material can be adequately carried out by a known method. For instance, coating can be carried out by mixing an adequate solvent such as (but not limited to) a commercially available manicure solution, a glass ionomer luting cement, or the like and a fluorescent material to prepare a paint and applying the paint to the clip surface. The fluorescent material concentration in a paint is not particularly limited. However, the paint contains fluorescent material at a concentration of preferably 0.001 to 20 mg/ml, more preferably 0.01 to 10 mg/ml, and further preferably 0.05 to 7 mg/ml. The paint is preferably insoluble or poorly soluble in water or body fluids. The use of a paint that is insoluble or poorly soluble in water or body fluids prevents dissociation of a fluorescent from the surface of the fluorescent material applied to the surface of a clip. Alternatively, the fluorescent material itself or the paint is mixed with a component of a clip so as to allow the clip to contain the fluorescent material. The paint can be applied to any portion constituting a clip (such as the arm of a clip or the base of the arm of a clip) or the entire clip. Preferably, the paint is applied to the arm of a clip. The term “the arm of a clip” refers to a portion used for pinching or insertion into tissues or organs (FIG. 8 (A)). When the paint is applied to the arm of a clip, if the clip is fixed to the intestinal wall or the like, the arm coated with the paint is fixed at a position close to the serosal side. This allows detection of NIR fluorescence at a high intensity for observation from the serosal side, which is advantageous.

The clip of the present invention can be introduced into a living body using a microscope as in the cases of clips generally used in the medical field.

### (b) An Ink Solution Containing a Fluorescent Material

The ink solution of the present invention is obtained by mixing an adequate solvent with the above fluorescent material. Such solvent is insoluble or poorly soluble in water or body fluids and may have a certain degree of viscosity according to need. Since the solvent is insoluble or poorly soluble in water or body fluids and may have a certain degree of viscosity, the ink solution cannot easily be diffused when introduced into a living body or a biological organ, tissue, or cells. An example of such solvent is a commercially available manicure solution or surgical hydrogel, but examples are not limited thereto. The ink solution contains the fluorescent material at a concentration of preferably 0.001 to 20 mg/ml, more preferably 0.01 to 10 mg/ml, and further preferably 0.05 to 7 mg/ml. In addition, it may contain a coloring pigment or a coloring dye according to need.

The ink solution of the present invention can be introduced into a living body or a biological organ or tissue by endoscopic injection as in the case of a tattoo injection that is generally used in the medical field.

(c) A Probe to which a Fluorescent Material is Bound

The probe of the present invention is a probe capable of recognizing a particular biomolecule, to which the above fluorescent material is bound. The term “recognizing” used herein refers to a situation in which the probe binds selectively and preferably specifically to a particular target biomolecule. Examples of such “biomolecule” include, but are not particularly limited to, DNA, RNA, a polypeptide, a peptide fragment, sugar, and a lipid that are highly expressed, overexpressed, or specifically expressed in a particular disease. Such “disease” is, for example, cancer, and particularly preferably solid cancer. Examples of solid cancer include, but are not limited to, lung cancer, esophageal cancer, breast cancer, gastric cancer, liver cancer, gallbladder/bile duct cancer, pancreatic cancer, colon/rectal cancer, bladder cancer, prostate cancer, and uterine cancer. The “probe” can be DNA, RNA, PNA, an antibody, an antibody fragment, a peptide, a compound, or the like which can recognize the above biomolecule. An example of such probe is a cyclic arginine-glycine-aspartic acid (RGD) peptide that can selectively bind to integrin αᵥβ₃ that is overexpressed in a variety of cancers (e.g., glioblastoma, melanoma, breast cancer, ovarian cancer, and prostate cancer).

A fluorescent material can bind directly or indirectly to a probe via a covalent bond or a non-covalent bond. For instance, binding of a fluorescent material and the RGD peptide can be carried out by reacting a maleimide-modified fluorescent material with a thiol-modified RGD peptide by a known method (Zako, T. et al., Biochem Biophys Res Commun 381, 54-8 (2009)).

The probe can be introduced into a living body by oral administration or parenteral administration (e.g., intravenous administration, intraarterial administration, local administration by injection, intraperitoneal or intrathoracic administration, subcutaneous administration, intramuscular administration, sublingual administration, percutaneous absorption, or intrarectal administration).

In addition, the probe can be formed in an adequate dosage form depending on the administration route. Specifically, the probe can be prepared in the following dosage forms: parenteral injection, suspension, capsules, granules, powder, pills, fine grains, troches, an agent for rectal administration, oleaginous suppository, and water-soluble suppository.

A variety of formulations of the probe can be produced using generally used excipients, extenders, binders, wetting-out agents, disintegrators, surfactants, lubricants, dispersants, buffers, preservatives, dissolution adjuvants, antiseptics, colorants, flavors, and stabilizers by conventional methods.

The amount of a probe contained in a formulation can vary according to the age, body weight, severity, and other conditions of a subject of administration. The amount thereof can be from 0.0001 mg to 100 mg/kg (body weight) per administration.

The bioimaging system of the present invention comprises at least (i) to (iv) described below:

(i) the above marker which is introduced into a living body;

(ii) a light source for irradiating the marker with NIR excitation light at 780 to 1700 nm;

(iii) a photographing means for detecting NIR fluorescence at 1000 to 2000 nm emitted from the marker excited by the light source, thereby obtaining image data; and

(iv) an image displaying means for displaying an observation image of image data obtained by the photographing means.

Components of the bioimaging system of the present invention are those that can be generally used in the optical field, the electronic material field, the medical field, the display device/display field, the optical communication field, the information communication field, and the like.

The “light source” may be a light source that can emit NIR excitation light at 780 to 1700 nm, preferably 900 to 1000 nm, and particularly preferably 980 nm for excitation of the marker and specifically of the fluorescent material. Examples of light source that can be used include: a variety of laser light sources (e.g., ion lasers, dye lasers, and semiconductor lasers); a variety of lamps such as high-pressure mercury lamps, low-pressure mercury lamps, ultrahigh-pressure mercury lamps, metal halide lamps, halogen lamps, nitrogen lamps, and xenon lamps; and a variety of LEDs. If necessary, the light source may have a different optical filter in order to achieve the optimal excitation wavelength.

The term “photographing means” refers to a means for creating fluorescence image data that constitute an observation image by detecting NIR fluorescence at 1000 to 2000 nm, preferably 1430 to 1670 nm, and more preferably 1550 nm emitted by the excited fluorescent material. A means having such functions can be adequately used. Examples of such photographing means include CCD cameras and CMOS cameras. Image data may be created as still image data or moving image data. The photographing means may comprise different types of optical filters for selectively detecting NIR fluorescence at 1000 to 2000 nm, preferably 1430 to 1670 nm, and more preferably 1550 nm. In addition, the photographing means may comprise a surgical laparoscope.

The term “image displaying means” refers to a means for displaying image data output from a photographing means in the form of an observation image. Examples of such image displaying means include CRT displays, liquid crystal displays, organic EL displays, plasma displays, and projection displays. A person who carries out the present invention can obtain a desired observation image by adequately adjusting the amount of light in a preferable manner while viewing an observation image displayed by an image displaying means.

In addition, the bioimaging system of the present invention can further comprise a means generally used in the field of fluorescence imaging such as a recording means for recording image data photographed by a photographing means, a reflection board for irradiating a subject with excitation light from a light source, and a laser scanner.

Further, the present invention relates to a method for detecting a lesion in a living body using the above bioimaging system. The method comprises the following steps of:

(a) positioning a marker comprising a fluorescent material at the site of a lesion and/or in the vicinity of a lesion in a living body;

(b) irradiating the marker with NIR excitation light from a light source from outside a living body or an organ or tissue of a living body; and

(c) detecting NIR fluorescence emitted from the excited fluorescent material.

According to the present invention, the term “living body” covers the living body of a human or a non-human animal and the organs and tissues thereof, unless otherwise specified.

The terms “organ” and “tissue” are not particularly limited. Examples of an “organ” include the lung, esophagus, breast, stomach, liver, gallbladder, bile duct, pancreas, colon, rectum, bladder, prostate gland, and uterus. Examples of “tissue” include tissue of any such organ.

Further, such “organ” or “tissue” may be not only an in vivo organ or tissue but also an in vitro organ or tissue.

In the present invention, the term “lesion” is not particularly limited. However, the term preferably refers to cancer and particularly preferably refers to solid cancer. Examples of such cancer include lung cancer, esophageal cancer, breast cancer, gastric cancer, liver cancer, gallbladder/bile duct cancer, pancreatic cancer, colon/rectal cancer, bladder cancer, prostate cancer, and uterine cancer.

A method for positioning a marker at the site of a lesion and/or in the vicinity of a lesion can be adequately selected depending on the form of the marker as described above.

Specifically, if a marker is in the form of a clip as described above, a single marker or a plurality of markers can be positioned at the site of a lesion and/or in the vicinity of a lesion (e.g., on the mucosal layer of the intestine) using an endoscope, as with generally used endoscopic clips. If a marker is in the form of an ink solution as described above, an ink solution can be injected into a single site or plurality of sites in a lesion and/or in the vicinity of a lesion (e.g., the submucosal layer of the intestine) using an endoscope, as with generally used tattoo injection. If a marker is in the form of a probe as described above, a probe is orally or parenterally administered (e.g., intravenous administration, intraarterial administration, local administration by injection, intraperitoneal or intrathoracic administration, subcutaneous administration, intramuscular administration, sublingual administration, percutaneous absorption, or intrarectal administration). Thus, the probe binds to a protein or nucleic acid that is specifically expressed or overexpressed in a lesion such that the probe can be positioned at the site of a lesion and/or in the vicinity of the lesion.

In any case, it is preferable to position a marker with a minimally invasive operation using an endoscope or injection regardless of the selected marker form.

The site of a marker in a living body (i.e., the lesion site) can be determined by irradiating a marker positioned in a living body with NIR excitation light at 780 to 1700 nm, preferably 900 to 1000 nm, and more preferably 980 nm from outside the living body or an organ or tissue of the living body (from the serosal side) and detecting NIR fluorescence emitted by a fluorescent material contained in the marker at 1000 to 2000 nm, preferably 1430 to 1670 nm, and more preferably 1550 nm.

A clip used in the method of the present invention differs from endoscopic clips that have been conventionally used as markers in that the clipping site can be clearly determined using NIR light from outside a living body or an organ or tissue of the living body (from the serosal side). In addition, the ink solution used in the method of the present invention has lower diffusivity than a solution conventionally used as a marker for tattoo injection. The site of injection with the ink solution also can be clearly determined using NIR light from outside a living body or an organ or tissue of the living body (from the serosal side). Further, a probe used in the method of the present invention specifically binds to a lesion. The lesion site can be clearly determined using NIR light from outside a living body or an organ or tissue of the living body (from the serosal side).

Accordingly, the lesion site can be determined in a noninvasive or minimally invasive manner by detecting a lesion by the method of the present invention. Therefore, follow-up observation of a lesion can be carried out in a noninvasive or minimally invasive manner. In addition, in the case of surgery for the removal of a lesion, the resection area can be minimized, achieving reduction of burdens imposed on patients.

Further, the present invention relates to a method for diagnosing a disease using the above bioimaging system. The method comprises the following steps of:

(a) administering a fluorescent-material-bound probe capable of binding to a particular protein or nucleic acid specifically expressed or overexpressed in a lesion to a subject;

(b) irradiating the probe with NIR excitation light from a light source from outside the body of the subject or an organ or tissue thereof;

(c) detecting NIR fluorescence emitted from the excited fluorescent material, thereby determining the occurrence or nonoccurrence of localized NIR fluorescence emission; and

(d) determining that the subject has the relevant disease if localized NIR fluorescence emission is detected in a particular organ and/or tissue.

In the present invention, the term “subject” covers animals such as humans and non-human animals, preferably mammals, and more preferably humans.

As described above, the probe of the present invention binds to a particular protein or nucleic acid specifically expressed or overexpressed in a lesion. First, the probe is orally or parenterally administered (intraocular, intrarectal, intraoral, local, intranasal, ocular instillation, intramuscular, intracavernous (bolus administration or injection), intracerebral, transdermal administration or the like) to a subject. After the elapse of a sufficient period of time (e.g., 0.5 to 24 hours, 1 to 12 hours, 1 to 6 hours, or 1 to 3 hours) during which the probe can bind to a particular protein or nucleic acid that is specifically expressed or overexpressed in a lesion (if any), the probe is irradiated with NIR excitation light at 780 to 1700 nm, preferably 900 to 1000 nm, and more preferably 980 nm from outside the subject (living body) or an organ or tissue thereof (from the serosal side). Then, localized NIR fluorescence emission at 1000 to 2000 nm, preferably 1430 to 1670 nm, and more preferably 1550 nm from the fluorescent material bound to the probe is detected at the corresponding site. If localization is observed, it can be judged that there is a high probability that the subject has the disease.

Thus, the presence or absence of a disease and a lesion area can be determined by the method of the present invention, allowing diagnosis or determination regarding the prognosis of the disease. In addition, since the method of the present invention can be carried out in a noninvasive or minimally invasive manner, burdens imposed on patients can be reduced.

The present invention is hereafter described in greater detail with reference to the following examples, although the present invention is not limited thereto.

### EXAMPLES

### Preparation of NIR Biophotonic Nanoparticle

A fluorescent material was prepared by a known technique used for preparation of an upconversion nanoparticle; that is to say, the homogenous precipitation method (Venkatachalam, N. et. al., Journal of the American Ceramic Society 92, 1006-1010, (2009)). Specifically, 20 mmol/L Y (NO₃)₃, 0.2 mmol/L Yb (NO₃)₃, and 0.2 mmol/L Er (NO₃)₃ were dissolved in purified water (200 mL), mixed with a 4 mol/L urea solution (100 mL), and stirred at 100° C. for 1 hour. The obtained precipitate was separated by centrifugation and dried at 80° C. for 12 hours. The thus obtained precursor was calcinated at 1200° C. for 60 minutes in an electric furnace. Accordingly, anhydrous crystalline Y₂O₃ nanoparticle codoped with anhydrous crystalline Yb and Er (hereafter referred to as “Y₂O₃:YbEr-NP”) was obtained.

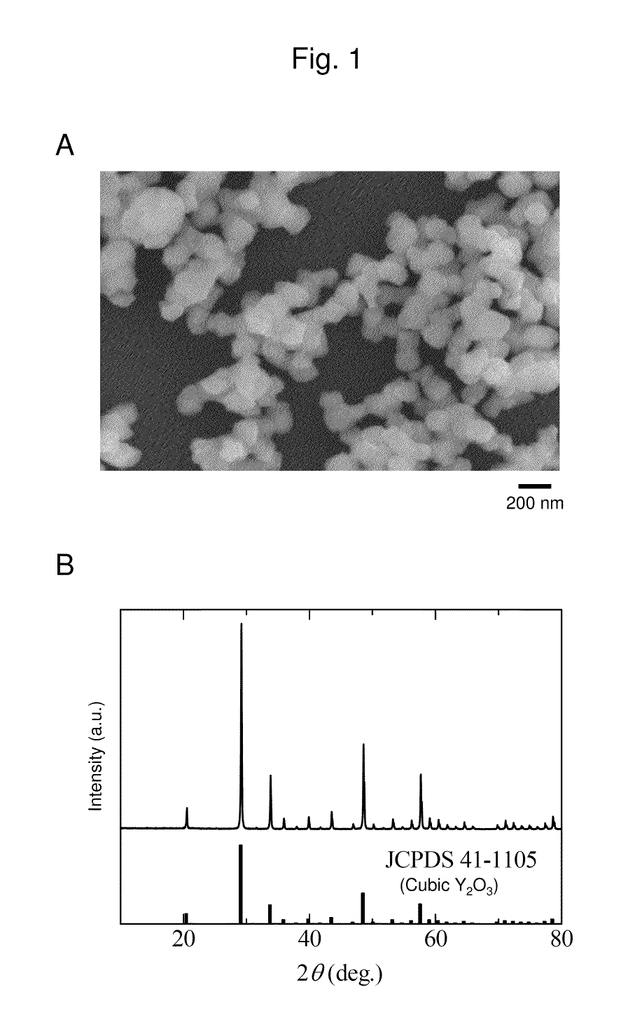

The obtained Y₂O₃:YbEr-NP was identified using a field emission scanning electron microscope (FE-SEM) and X-ray diffraction (XRD). FIGS. 1 (A) and (B) show FE-SEM analysis and XRD results, respectively. The Y₂O₃:YbEr-NP particle size was approximately 130±25 nm. Based on the XRD pattern, the obtained Y₂O₃:YbEr-NP was confirmed to be single-phase Y₂O₃:YbEr-NP because all peaks were identified as cubic Y₂O₃ (JCPDS 41-1105)-derived peaks.

### (Optical Absorption and Fluorescence of Y₂O₃:YbEr-NP)

The optical absorption spectrum of Y₂O₃:YbEr-NP was analyzed by a known technique using a spectrometer equipped with an integrating sphere (U-4000, Hitachi). In addition, the fluorescence spectrum of Y₂O₃:YbEr-NP was recorded by a known technique using a spectrometer (AvaSpec-NIR256-1.7, Avantes) with 980-nm excitation light and a laser diode (LD, SLI-CW-9MM-C1-980-1M-PD, Semiconductor Laser International Corp.).

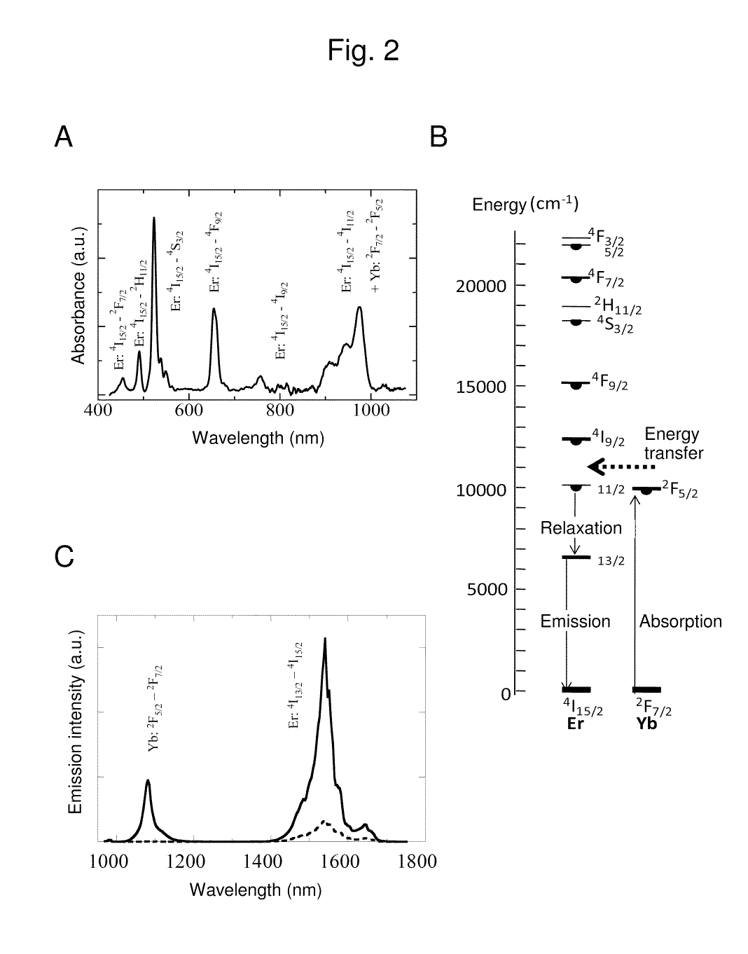

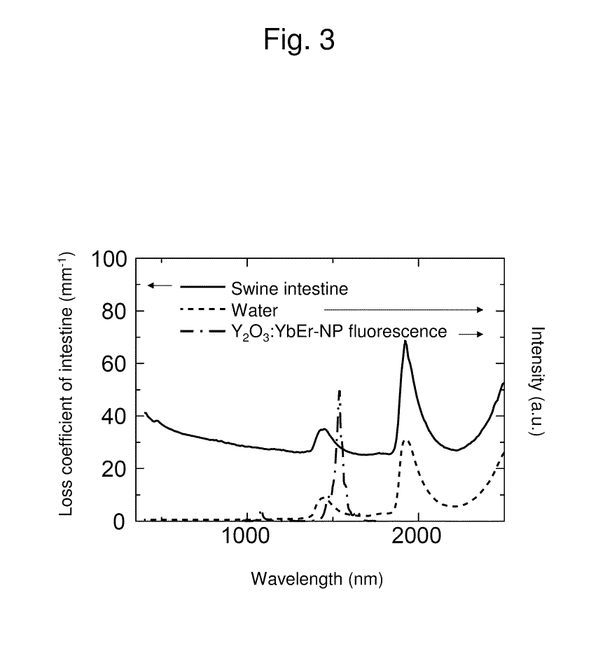

FIG. 3 shows results of analysis of the optical absorption spectrum and the fluorescence spectrum of Y₂O₃:YbEr-NP. In this experiment, Yb³⁺ was added as a so-called “sensitizer” for increasing the absorption efficiency of excitation light at 980 nm. FIG. 2 (A) shows the absorption spectrum. As is apparent from the results, a strong absorption band of Yb³⁺ was observed. The absorbed excitation light at 980-nm was mainly absorbed by Yb³⁺ and the excitation energy was transferred to Yb³⁺, resulting in emission of NIR fluorescence at 1550 nm (FIG. 2 (B)). Also in this experiment, the fluorescence spectrum of Y₂O₃:Er-NP used as a control was analyzed as in the case of Y₂O₃:YbEr-NP. As is apparent from FIG. 2 (C), the NIR emission of Y₂O₃:YbEr-NP is much higher than that of Y₂O₃:Er-NP, indicating that NIR fluorescence can be enhanced by codoping of Y₂O₃ with Yb³⁺ and Er³⁺.

Next, the loss spectrum for a swine intestine was analyzed with the system used for the optical absorption spectral analysis described above. A slice of the swine intestine (thickness: 250-330 μm) was sandwiched between two glass slides. The loss spectrum was determined in a normal mode without using the integrating sphere.

FIG. 3 shows results of analysis of the optical absorption loss spectrum for the swine intestine. The spectrum was obtained in the following manner. Two swine intestine sections having different thicknesses of 330 μm and 220 mm, respectively, were subjected to spectral measurement. The spectrum for the section with a thickness of 220 μm was subtracted from the spectrum for the section with a thickness of 330 μm. Thus, the net optical absorption loss due to a thickness difference of 110 μm was obtained. In this way, the influence of surface reflection can be ignored. In addition, the net loss value proportional to thickness in a test sample can be obtained, making it possible to evaluate test samples having different thicknesses by the multiplication of the value designating a given thickness.

The spectrum was divided in accordance with the corresponding thickness to obtain a coefficient spectrum. In FIG. 3, the absorption spectrum of water and the Y₂O₃:YbEr-NP fluorescence spectrum were coplotted.

There are absorption band peaks at 1420 nm, which are derived from the second harmonic absorption of the O—H stretching vibration in water molecules. The Y₂O₃:YbEr-NP fluorescence spectrum overlaps the absorption band of the intestine and that of water. However, the tail of the fluorescence spectrum is not within the absorption bands, indicating that the fluorescence spectrum can be observed through the intestinal wall.

### (NIR Imaging Inside the Swine Intestine)

A tablet having a diameter of 3 mm and a length of 6 mm was prepared by mixing Y₂O₃:YbEr-NP and a dental composite resin (Fuji I, GC).

An NIR imaging system was composed of the following:

a fiber pigtail laser diode (2 W) (LU0975T050, Lumics, Berlin, Germany) (for a 980-nm excitation light source);

a laser scanner (VM500+, GSI Group) (for planerirradiation of excitation light); and

an InGaAs CCD camera (NIR-300PGE, VDS Vosskuehler, Osnabrueck, Germany) (for detection of NIR fluorescence between 1100- to 1600-nm).

The Y₂O₃:YbEr-NP tablet was introduced into an excised swine intestine sample (hereafter referred to as “swine intestine sample”). The swine intestine was irradiated from the serosal side with NIR excitation light at 980 nm using an NIR imaging system. Accordingly, NIR fluorescence was detected at 1550 nm.

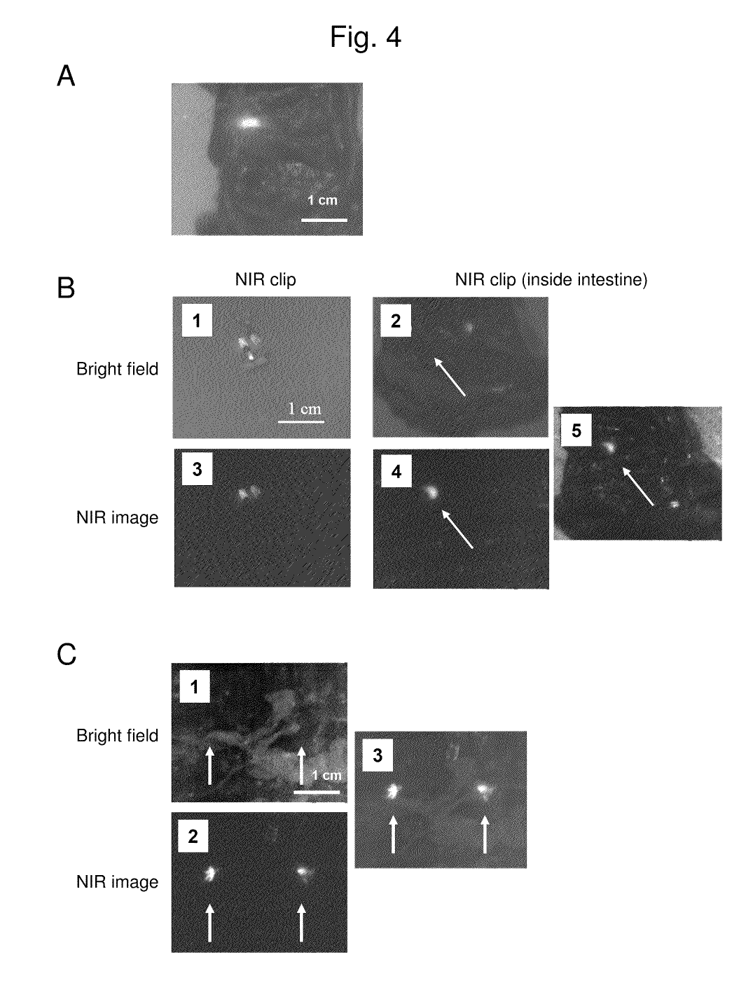

FIG. 4 (A) shows an NIR image of the Y₂O₃:YbEr-NP tablet introduced into the swine intestine sample. Fluorescence emitted from the Y₂O₃:YbEr-NP tablet was clearly detected from the serosal side through the intestinal wall. The results indicate that NIR excitation light and Y₂O₃:YbEr-NP-derived NIR fluorescence have sufficient intensity to penetrate the intestinal wall.

### (Y₂O₃:YbEr-NP-Coated Endoscopic Clip (1))

The base of the arm of a known endoscopic clip (OLYMPUS) (Raju, G. S. et. al., Gastrointest Endosc 59, 267-79 (2004)) was coated with a paint containing Y₂O₃:YbEr-NP such that a Y₂O₃:YbEr-NP-coated endoscopic clip (hereafter referred to as an “NIR clip (1)”) was prepared.

The NIR clip was fixed to the inner wall of the swine intestine sample (i.e., the mucosal side). The NIR clip (1) was detected from outside the swine intestine sample (i.e., the serosal side) using the NIR imaging system in the manner described above.

FIG. 4 (B) shows the results. The results indicate that the NIR fluorescence emitted from the NIR clip (1) upon NIR excitation has sufficient intensity to penetrate the intestinal wall. Although the surface of the base of the NIR clip (1) was coated with Y₂O₃:YbEr-NP to a thickness of only several tens of micrometers, the intensity of NIR fluorescence emitted by the NIR clip (1) was found to be sufficient and comparable to that of NIR fluorescence emitted by the tablet.

The results indicate that an NIR clip (1) can replace endoscopic clips that have been conventionally used for marking for surgery or other purposes.

### (Y₂O₃:YbEr-NP-Containing Ink Solution)

A Y₂O₃:YbEr-NP-containing solution (hereafter referred to as an “NIR ink solution”) was prepared by disrupting Y₂O₃:YbEr-NP in a manicure solution using a mortar and a pestle, followed by mixing.

An NIR ink solution was injected into the inner wall of the swine intestine sample (i.e., the mucosal side). The NIR ink solution was detected from outside the swine intestine sample (i.e., the serosal side) using the NIR imaging system in the manner described above.

FIG. 4 (C) shows the results. NIR fluorescence emitted from the NIR ink solution upon NIR excitation was detected at a sufficient intensity from outside the swine intestine sample (i.e., the serosal side). The results indicate that injection of an NIR ink solution can replace tattoo injection conventionally used for marking for surgery or other purposes.

### (Y₂O₃:YbEr-NP-Bound Probe)

Y₂O₃:YbEr-NP (particle diameter: 50-200 nm) was bound to a cyclic arginine-glycine-aspartic acid (RGD) peptide via PEG by a conventionally known method (Zako, T. et. al., Biochem Biophys Res Commun 381, 54-8, (2009)). Thus, PEG-RGD-modified Y₂O₃:YbEr-NP was produced (RGD-PEG-Y₂O₃:YbEr-NP). Specifically, the Y₂O₃:YbEr-NP (50 mg) were suspended in 45 mL of 2-propanol and subjected to ultrasonication. After 300 μl of 3-aminopropyltrimethoxysilane (APTES) was added, the mixture was stirred for 24 h at 70° C. The particles were then isolated, washed five times with ethanol by centrifugation, and finally dried in air at room temperature. The APTES-modified Y₂O₃:YbEr-NP (APTES-Y₂O₃:YbEr-NP) (20 mg) were suspended in 10 mL of dry-dimethyl sulfoxide (DMSO, Wako, Tokyo, Japan), to which was added 500 μM heterofunctional PEG containing N-hydroxysuccinimide (NHS) and maleimide (MA) at the both ends (NHS-PEG-MA) (MW=5000, Sunbright MA-050HS, NOF Corp., ToKyo, Japan) and stirred for 24 h at room temperature. The MA-PEG modified APTES-Y₂O₃:YbEr-NP (MA-PEG-Y₂O₃:YbEr-NP) were isolated, washed three times with dry DMSO by centrifugation, and suspended in 10 mL of dry DMSO.

In order to introduce a thiol group into a cyclo(RGDyK) peptide (potent integrin αᵥβ₃ antagonist), 1 mg of cyclo(RGDyK) was dissolved in 500 μL of dry DMSO, to which was added 1 mg of S-acet-ylthioglycolic acid N-hydroxysuccinimide ester (SATA), and stirred over night at room temperature. Then, 1 mL of 10% hydroxylamine was added and stirred for 3 h to deprotect a thiol group and to yield the thiolated RGD peptide cyclo(RGDy(ε-acetylthiol)K), denoted as RGD-SH. The MA-PEG-Y₂O₃:YbEr-NP was allowed to react with RGD-SH for 12 h at room temperature in dry DMSO. The final conjugate (RGD-PEG-Y₂O₃:YbEr-NP) was isolated, washed three times with distilled water by centrifugation.

U87MG (high integrin αᵥβ₃ expression) glioblastoma cells were purchased from European Collection of Cell Cultures. U87MG cells were grown in E-MEM medium with 10% FBS, 1% NEAA, 1% sodium pyruvate and 1% penicillin-streptomycin in 5% CO₂ at 37° C. Cells were detached from cell culture dish with trypsin-EDTA for passage. Cells were plated in 35 mm dish at a density of 40,000 cells/mL. Cells were then incubated in 2.0 mL medium in the presence of 10 μg/mL RGD-PEG-Y₂O₃:YbEr-NP for 3 h. Cells were washed three times with distilled water, and then 2 mL of medium was added. Thereafter, RGD-PEG-Y₂O₃:YbEr-NP was detected using the aforementioned NIR imaging system in the manner described above.

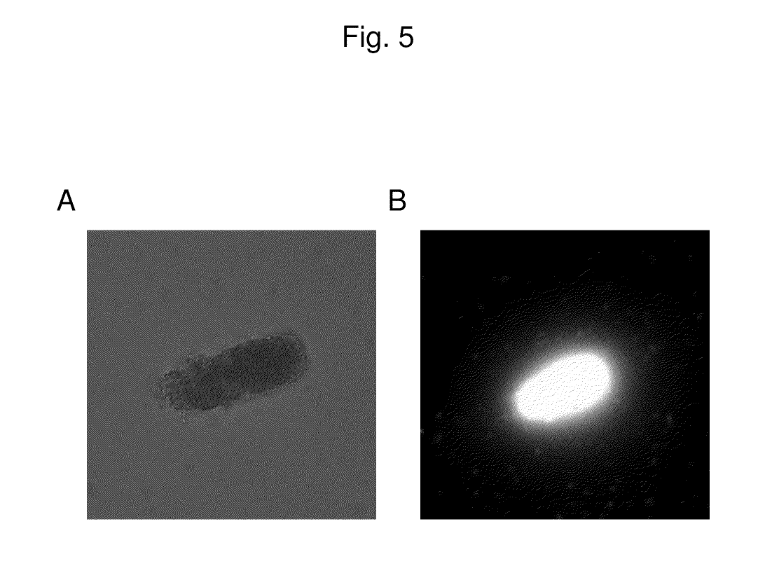

FIG. 5 shows the results. NIR fluorescence emitted from RGD-PEG-Y₂O₃:YbEr-NP was exclusively detected in U87MG cells upon NIR excitation.

The results indicate that Y₂O₃:YbEr-NP-bound probes can be used for cancer detection.

(NIR Imaging Inside a Swine Colon Sample with the Use of a Surgical Laparoscope)

The above Y₂O₃:YbEr-NP tablet was positioned outside or inside an excised swine colon sample (hereafter referred to as a “swine colon sample”) and the NIR image of the tablet was observed using a near-infrared camera to which a surgical laparoscope was connected.

The NIR imaging system comprising a surgical laparoscope used in this Example was composed of the following

a fiber pig-tailed laser diode (2 W) (LU0975T050, Lumics, Berlin, Germany) (for a 980-nm excitation light source);

a laser scanner (VM500+, GSI Group) (for surface irradiation with excitation light);

a surgical laparoscope (MACHIDA Endoscope Co., Ltd); and

an InGaAs CCD camera (Xeva USB 1.7 320 TE3, Xenics, Leuven, Beigium) (for detection of 1100- to 1600-nm NIR fluorescence).

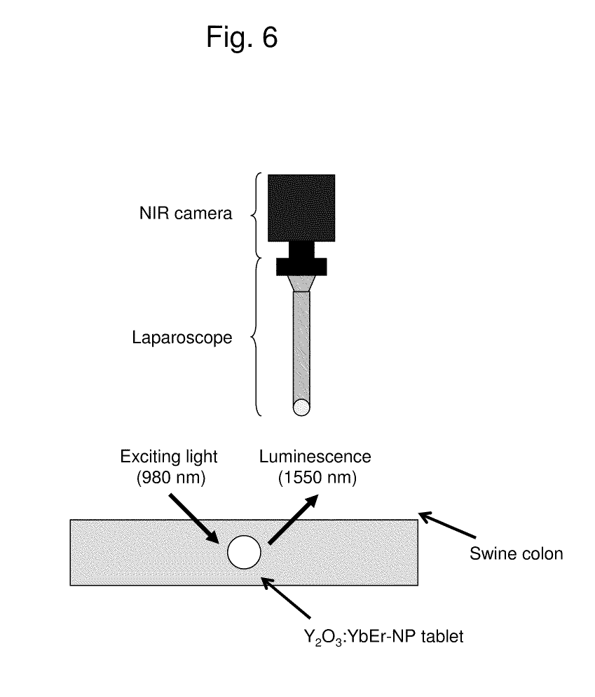

FIG. 6 schematically shows a near-infrared camera to which a surgical laparoscope is connected.

The Y₂O₃:YbEr-NP tablet was introduced into the swine colon sample. The Y₂O₃:YbEr-NP tablet was irradiated with NIR excitation light at 980 nm with the use of the NIR imaging system composed of a surgical laparoscope from outside the serosal membrane of the colon sample. As a result, NIR fluorescence emitted from the Y₂O₃:YbEr-NP tablet was detected at 1550 nm.

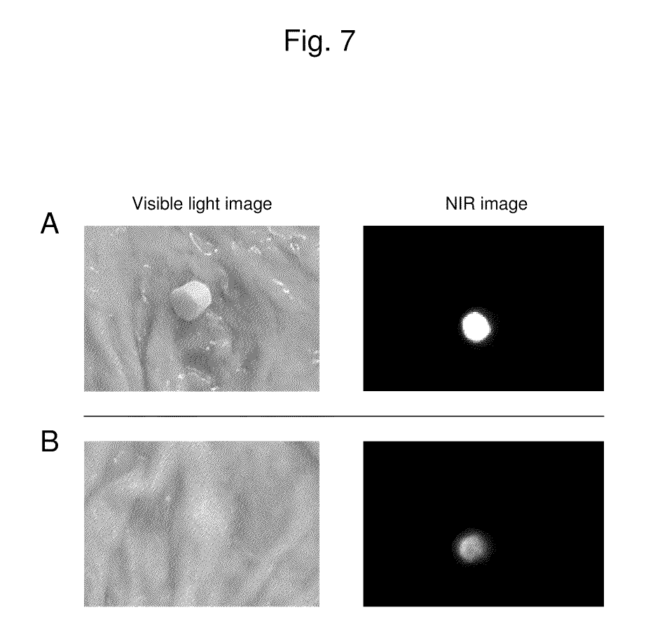

FIG. 7 (A) shows a visible light image and an NIR image of the Y₂O₃:YbEr-NP tablet positioned outside the swine colon sample and FIG. 7 (B) shows a visible light image and an NIR image of the Y₂O₃:YbEr-NP tablet positioned inside the swine colon sample. As is apparent from FIG. 7 (B), fluorescence emitted from the Y₂O₃:YbEr-NP tablet was clearly detected through the intestine wall from outside the serosal membrane. The results suggested that a Y₂O₃:YbEr-NP tablet positioned inside a swine colon sample can be clearly detected using an NIR imaging system composed of a surgical laparoscope.

### (Y₂O₃:YbEr-NP-Coated Endoscopic Clip (2))

The arm of the known endoscopic clip described above was coated with a paint containing Y₂O₃:YbEr-NP such that a Y₂O₃:YbEr-NP-coated endoscopic clip was produced (hereafter referred to as an “NIR clip (2)”) (FIG. 8 (A)). Such NIR clip (2) is obtained by coating the arm of an endoscopic clip with a paint containing Y₂O₃:YbEr-NP. When the clip is fixed inside an intestine, the arm is fixed to the intestinal wall. Therefore, in such case, the Y₂O₃:YbEr-NP-coated arm can be fixed at a position closer to the serosal side than the position of the base of the arm of the NIR clip (1) coated with a paint containing Y₂O₃:YbEr-NP (FIG. 8 (B)).

For coating of the NIR clip (2) (endoscopic clip), Y₂O₃:YbEr-NP was mixed with a solution for a glass ionomer luting cement (GC). A glass ionomer luting cement powder was added thereto. The ratio of Y₂O₃:YbEr-NP and cement solution was 1:2. The end of the arm of the clip was coated with the solution and allowed to stand still. It was necessary to devise a way to coat a endoscopic clip with a small amount of the Y₂O₃:YbEr-NP particle solution so as to allow reattachment of the clip to an endoscopy. The size of the fixed cement should be within 1 mm, so as to allow the coated clip to be reattached in the endoscopy.

Each of the NIR clip (2) and the NIR clip (1) was fixed to the inner wall of a swine colon sample (i.e., the mucosal side) and detected from outside the swine colon sample (i.e., the serosal side) with the use of the NIR imaging system comprising a surgical laparoscope. For detection, a 50-mL tube was inserted into each colon sample so as to make a hollow space therein.

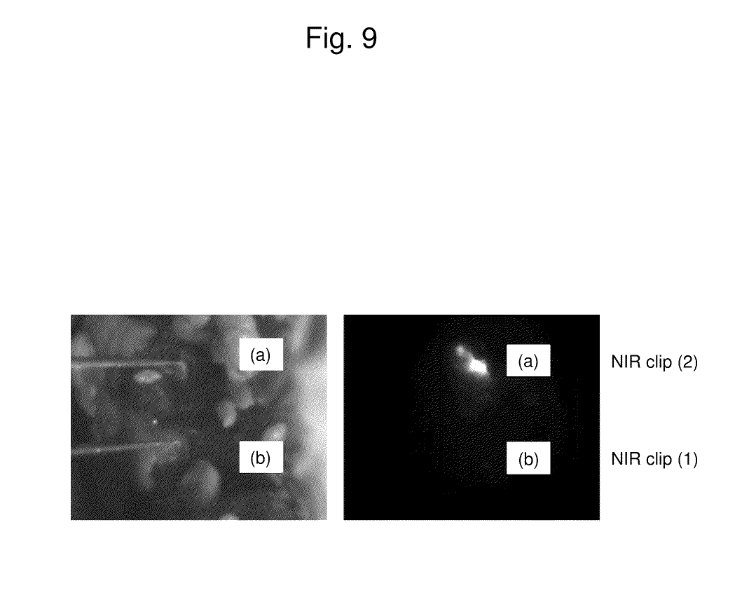

FIG. 9 shows the results. When the NIR clip (2) was fixed inside the intestine, the Y₂O₃:YbEr-NP-coated arm of the clip was fixed to the intestinal wall, allowing to fix the Y₂O₃:YbEr-NP coat at a position close to the serosal side. Accordingly, it was possible to detect NIR fluorescence at an intensity (FIG. 9 (a)) greater than that detected in the case of the NIR clip (1) (FIG. 9 (b)).

### (Surgical Simulation Experiment Using a Swine Colon Sample)

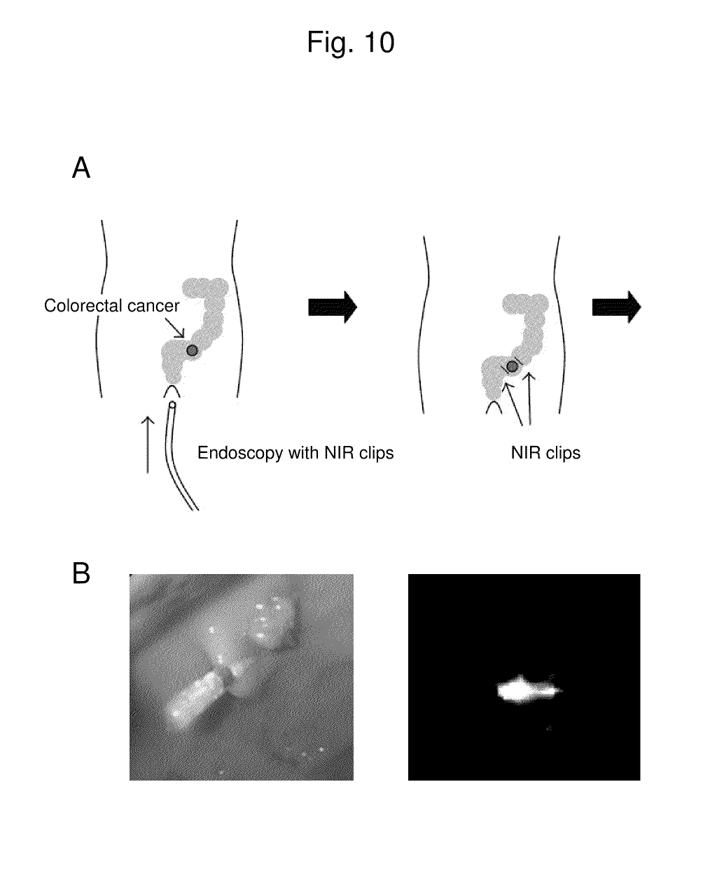

The NIR clip (1) was fixed inside the colon of a pig via the transanal route with the use of an endoscopy by a conventionally known method (FIG. 10 (A)). NIR fluorescence was detected using the NIR imaging system comprising a surgical laparoscope in the manner described above.

As a result, the image of the NIR clip fixed to the internal membrane of the colon was successfully obtained from the serosal side using a near-infrared camera to which a surgical laparoscope was connected (FIG. 10 (B)).

The bioimaging marker of the present invention can emit NIR fluorescence that can sufficiently penetrate a living body upon excitation with NIR excitation light that can sufficiently penetrate a living body. Therefore, the position of the bioimaging marker can be easily detected from outside a living body even if the marker is introduced into the living body. Thus, the bioimaging marker of the present invention is very useful for marking of a given site in a living body and a lesion. Therefore, the bioimaging marker of the present invention can be expected to be used for a novel bioimaging system or method that is very useful in the field of biomedical research and is also very useful for disease diagnosis, prognosis diagnosis, and surgery.

1. A bioimaging marker comprising a fluorescent material obtained by doping a ceramic with one or more rare earth ions and/or one or more elemental ions selected from the group consisting of uranium (U), titanium (Ti), chromium (Cr), nickel (Ni), manganese (Mn), molybdenum (Mo), rhenium (Re), and osmium (Os) ions, wherein the marker is in the form of any one of the following (a) to (c):
(a) a clip comprising a fluorescent material;
(b) an ink solution containing a fluorescent material; or
(c) a probe capable of recognizing a particular biomolecule to which a fluorescent material is bound, and wherein
the marker emits near-infrared fluorescence at 1000 to 2000 nm when irradiated with near-infrared excitation light at 780 to 1700 nm.

2. The marker according to claim 1, wherein the clip comprise the fluorescent material in the arm.

3. The marker according to claim 1 or 2, wherein the fluorescent material is in the form of a nanoparticle of yttrium oxide obtained by codoping of Y₂O₃ with ytterbium (Yb) ion and erbium (Er) ion.

4. The marker according to claim 3, which emits near-infrared fluorescence at 1430 to 1670 nm when irradiated with near-infrared excitation light at 900 to 1000 nm.

5. A bioimaging system for visualizing a marker introduced into a living body with the use of near-infrared light, which comprises at least the following (i) to (iv):
(i) the marker according to claim 1, which is introduced into a living body;
(ii) a light source for irradiating the marker with near-infrared excitation light at 780 to 1700 nm from outside a living body;
(iii) a photographing means for detecting near-infrared fluorescence at 1000 to 2000 nm emitted from the marker excited by the light source, thereby obtaining image data; and
(iv) an image displaying means for displaying an observation image of image data obtained by the photographing means.

6. The system according to claim 5, wherein the marker is irradiated with near-infrared excitation light at 900 to 1000 nm.

7. The system according to claim 5 or 6, wherein the photographing means detects near-infrared fluorescence emitted from the marker at 1430 to 1670 nm.

8. A bioimaging method using a marker introduced into a living body of an animal wherein the bioimaging system according to claim 5 is used, which comprises the following steps of:
(a) introducing a marker into a living body of an animal;
(b) irradiating the marker from outside the living body with near-infrared excitation light from a light source; and
(c) detecting near-infrared fluorescence emitted from the excited fluorescent material by a photographing means.

9. A bioimaging method using a marker introduced into a human organ or tissue wherein the bioimaging system according to claim 5 is used, which comprises the following steps of:
(a) irradiating a marker introduced into a human organ or tissue with near-infrared excitation light from a light source from outside the human organ or tissue; and
(b) detecting near-infrared fluorescence emitted by the excited fluorescent material by a photographing means.
